(19) United States
(12) Patent Application Publication
(10) Pub. No.: US 2024/0382754 A1
(43) Pub. Date: Nov. 21, 2024

(54) FASCICULAR MAPPER FOR ENHANCING INTRANEURAL ELECTRODES IMPLANTATION SPECIFICITY

(71) Applicant: BOARD OF TRUSTEES OF THE UNIVERSITY OF ARKANSAS, Little Rock, AR (US)

(72) Inventors: Anil K. Thota, Bentonville, AR (US); Ranu Jung, Fayetteville, AR (US)

(73) Assignee: BOARD OF TRUSTEES OF THE UNIVERSITY OF ARKANSAS, Little Rock, AR (US)

(21) Appl. No.: 18/667,296

(22) Filed: May 17, 2024

Related U.S. Application Data
(60) Provisional application No. 63/467,261, filed on May 17, 2023.

(51) Int. Cl. A61N 1/36; A61B 5/383; A61B 5/395; A61N 1/05

(52) U.S. Cl. CPC A61N 1/36103; A61B 5/383; A61B 5/395; A61N 1/0558; A61N 1/36014

(57) ABSTRACT

Embodiments of the present disclosure pertain to systems for locating a peripheral nerve, which include a plurality of electrodes operational to be actuated independently of other electrodes and base area anchoring the plurality of electrodes. Additional embodiments of the present disclosure pertain to methods of locating a fascicle or a portion of a fascicle of a peripheral nerve by (1) electrically stimulating different regions of the peripheral nerve; (2) detecting activity from the stimulated regions; and (3) correlating the detected activity from the stimulated regions to the presence of a fascicle or a portion of a fascicle at the stimulated regions. The methods of the present disclosure may also include steps of (4) generating a map of fascicles in the peripheral nerve and (5) implanting electrodes into the located fascicles.

### CROSS-REFERENCE TO RELATED APPLICATIONS

This application claims priority to U.S. Provisional Patent Application No. 63/467,261, filed on May 17, 2023. The entirety of the aforementioned application is incorporated herein by reference.

### STATEMENT REGARDING FEDERALLY SPONSORED RESEARCH

This invention was made with government support under R01-EB027584 and R01-EB023261, awarded by the National Institutes of Health. The government has certain rights in the invention.

### BACKGROUND

A need exists for more effective methods and systems for increasing intraneural electrode implantation specificity. Numerous embodiments of the present disclosure aim to address the aforementioned need.

### SUMMARY

In some embodiments, the present disclosure pertains to systems for mapping a peripheral nerve. In some embodiments, the systems of the present disclosure include a plurality of electrodes. In some embodiments, each of the plurality of electrodes is operational to be actuated independently of other electrodes. In some embodiments, the systems of the present disclosure also include a base area. In some embodiments, the base area anchors the plurality of electrodes.

Additional embodiments of the present disclosure pertain to methods of locating a fascicle or a portion of a fascicle of a peripheral nerve (e.g., a group of neural fibers within fascicles). In some embodiments, such methods include: (1) electrically stimulating different regions of the peripheral nerve; (2) detecting activity from the stimulated regions; and (3) correlating the detected activity from the stimulated regions to the presence of a fascicle or a portion of a fascicle at the stimulated regions. In some embodiments, the methods of the present disclosure also include generating a map of fascicles in the peripheral nerve. In some embodiments, the methods of the present disclosure also include a step of implanting electrodes into the located fascicles.

### BRIEF DESCRIPTION OF THE DRAWINGS

A better understanding of the present invention can be obtained when the following detailed description is considered in conjunction with the following drawings, in which:

FIGS. 1A-1B illustrate a system for locating a fascicle or a portion of a fascicle in accordance with various embodiments of the present disclosure.

FIGS. 1C-1D illustrate methods of locating a fascicle or a portion of a fascicle in accordance with various embodiments of the present disclosure.

FIGS. 2A-2D illustrate fascicular mapping, where two fascicular mapper (MAP) prototypes (FIGS. 2A and 2B) were fabricated utilizing a silicone vessel loop and a microwire electrode array to accommodate longitudinal intrafascicular electrode (LIFE) implantation with a custom-made platform (FIGS. 2C and 2D). A version of MAP (FIG. 2B) was placed on the nerve and connected to a neurostimulator for mapping (FIG. 2C). The functional output of fascicles or a portion of the fascicles were mapped by stimulating mapping electrodes one-by-one to activate the nerve fibers eliciting muscle contractions. As illustrated in FIG. 2D, target specific implantation of LIFEs guided by the functional mapping output from the mapping process enhances the specificity.

FIGS. 3A-3C show data related to the fascicle mapping capability of the MAP prototypes. Stimulation of mapping electrodes elicited either ankle flexion or extension. One electrode pair (FIG. 3A: M1M2) activated gastrocnemius muscle only, eliciting ankle extension. Two other electrode pairs (FIG. 3B: M1M3, and FIG. 3C: M2M3) activated gastrocnemius and tibialis anterior muscles, eliciting both ankle flexion or extension at different locations. The results show that the fascicle mapper can map the fascicles of the rat's sciatic nerve.

FIG. 4A-4D show additional data related to the fascicle mapping capability of the MAP prototypes. FIGS. 4A-4B show video frames and FIGS. 4C-4D show ankle angle profiles illustrating ankle extension and ankle flexion. To achieve ankle extension by contracting the gastrocnemius muscle (increasing angle from anatomical position (FIG. 4C)), the mapping electrode is interfaced with the tibial fascicle. To achieve ankle flexion by contracting the tibialis anterior muscle (decreasing angle from anatomical position (FIG. 4D)) the mapping electrode is interfaced with the peroneal fascicle.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Bioelectronic medicine, an emerging research field, promises to improve and restore health without the debilitating side effects of drugs. The primary foundations for this emerging field are the engineering and scientific knowledge gained from the past few decades of neuromodulation and pharmaceutical studies. Hence, the field of bioelectronic medicine is sometimes referred to as Electroceuticals™ because doses of electrical stimulation are delivered to treat diseases as pharmaceutical drugs do.

A primary focus of bioelectronic medicine is to selectively stimulate specific nerve fibers with electrical pulses (doses) to trigger the body's internal physiological response to fight diseases and conditions. Various types of neural electrodes referred to as peripheral neural interfaces (PNI) are used in neuromodulation studies to stimulate nerve fibers. A PNI with high selectivity and low specificity can activate a distinct group of nerve fibers but typically do not elicit the desired biological response. On the other hand, stimulation of nerve with low selectivity and low specificity PNI clicits not only desired (on-target) but also unwanted (off-target) biological responses.

A PNI with high selectivity and specificity plays a pivotal role in the success of bioelectronic medicine by activating a distinct group of nerve fibers to elicit a biological response to treat pathological conditions by creating disease-specific molecular mechanisms. The selectivity of a PNI can be increased by placing the electrodes near the target nerve fibers. Thus, the selectivity of the intrafascicular electrodes is higher than the nerve cuff electrodes because the former penetrates nerve fascicles to interface with a small group of nerve fibers directly.

Even with intrafascicular electrode placement in the nerve fascicle, achieving specificity can be difficult because multiple distinct groups of nerve fibers are present in a large fascicle. Furthermore, the anatomical somatotopy can vary between the proximal portions of the nerve and the distal portions.

Specificity can be increased by implanting multiple intrafascicular electrodes in proximity such that at least one electrode can be stimulated to activate the group of nerve fibers that elicit the desired function (on-target). At the same time, one or more other electrodes that elicit unwanted outcomes by undesirable neural fiber recruitment (off-target) are not selected for stimulation. Implantation of multiple intrafascicular electrodes can offer high specificity and selectivity, but often at the cost of high invasiveness.

The tradeoff between desirable properties of PNI and invasiveness can be achieved by advanced stimulation strategies. However, the advanced stimulation strategies require high computational costs, such as additional time to program stimulation parameters and the need for customized neurostimulation hardware.

Other methods of increasing electrode implantation specificity is to know the functional outcome of the nerve fibers before implanting electrodes. The functional mapping can be achieved by stimulating the nerve fibers to elicit muscle contractions or evaluating somatosensory evoked potentials. However, such processes suffer from limited specificity.

As such, a need exists for more effective methods and systems for increasing intraneural electrode implantation specificity. Numerous embodiments of the present disclosure aim to address the aforementioned need.

In some embodiments, the present disclosure pertains to systems for mapping a peripheral nerve. In some embodiments, the systems of the present disclosure include a plurality of electrodes. In some embodiments, each of the plurality of electrodes is operational to be actuated independently of other electrodes. In some embodiments, the systems of the present disclosure also include a base area. In some embodiments, the base area anchors the plurality of electrodes. In some embodiments, the electrodes are fully looped around the base area. In some embodiments, the electrodes are partially looped around the base area.

In some embodiments, the systems of the present disclosure also include a wiring system. In some embodiments, the wiring system connects the plurality of electrodes to a power source. In some embodiments, the power source includes a neural stimulator.

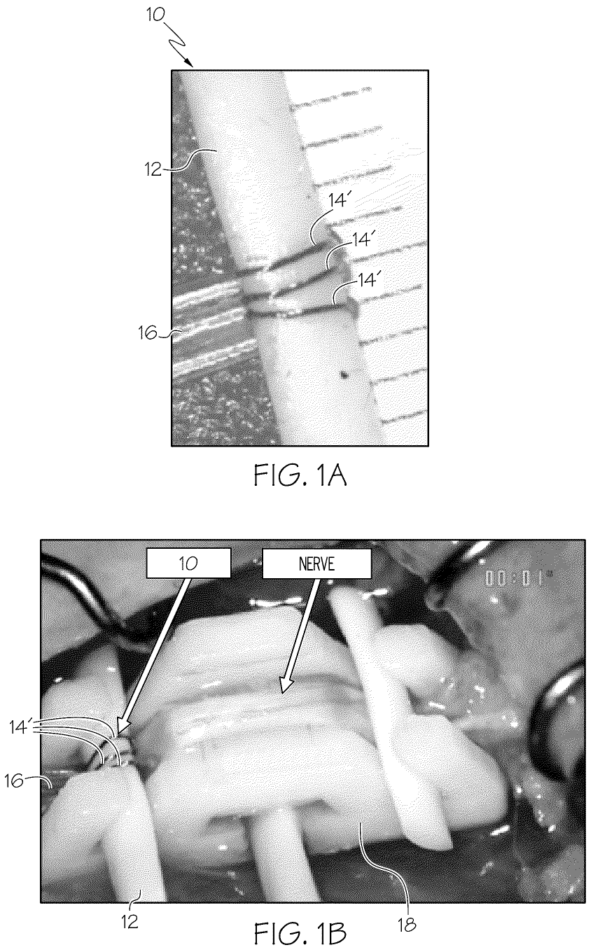

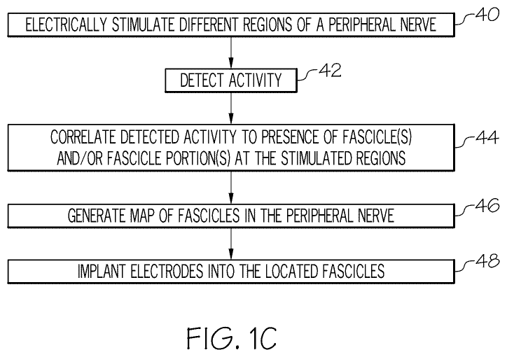

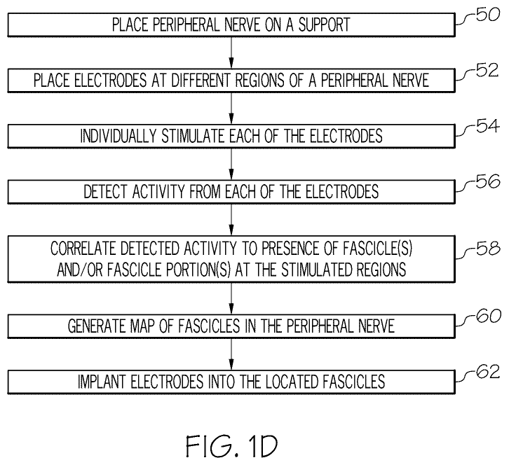

An example of a system of the present disclosure is illustrated in FIGS. 1A-1B as system 10. In this example, system 10 includes a plurality of electrodes 14. Electrodes 14 are in the form of an array, aligned parallel to one another, and operational to be actuated independently of one another. System 10 also includes a base area 12 that anchors electrodes 14, and a wiring system 16 that connects electrodes 14 to a power source. In this example, electrodes 14 are fully looped around base area 12. Additionally, electrodes 14 are associated with base area 12 in the form of an array.

Additional embodiments of the present disclosure pertain to methods of locating a fascicle or a portion of a fascicle of a peripheral nerve. In some embodiments illustrated in FIG. 1C, such methods include: electrically stimulating different regions of the peripheral nerve (step 40); detecting activity from the stimulated regions (step 42); and correlating the detected activity from the stimulated regions to the presence of a fascicle or a portion of a fascicle at the stimulated regions (step 44). In some embodiments, the methods of the present disclosure also include a step of generating a map of fascicles in the peripheral nerve (step 46). In some embodiments, the methods of the present disclosure also include a step of implanting electrodes into the located fascicles (step 48).

As set forth in more detail herein, the methods and systems of the present disclosure can have numerous embodiments. For instance, in some embodiments illustrated in FIG. 1D, the methods of the present disclosure include one or more of the following steps: placing a peripheral nerve on a support (step 50); placing electrodes at different regions of a peripheral nerve (step 52); individually stimulating each of the electrodes (step 54); detecting activity from each of the electrodes (step 56); correlating the detected activity from the stimulated regions to the presence of a fascicle or a portion of a fascicle at the stimulated regions (step 58); generating a map of fascicles in the peripheral nerve (step 60); and implanting electrodes into the located fascicles (step 62).

Additionally, in some embodiments, the systems of the present disclosure may be utilized to implement the methods of the present disclosure. For instance, in some embodiments illustrated in FIGS. 1A-1B, system 10 may be utilized in accordance with the methods of the present disclosure to locate a fascicle or a portion of a fascicle of a peripheral nerve. For instance, a peripheral nerve may first be placed on support 18. Thereafter, electrodes 14 may be placed at different regions of the peripheral nerve. Next, each of the electrodes 14 may be individually stimulated. The detected activity from the stimulated regions may then be correlated to the presence of a fascicle or a portion of a fascicle at the stimulated regions, which could then be utilized to generate a map of fascicles in the peripheral nerve. Furthermore, electrodes may be implanted into the located fascicles while the peripheral nerve remains immobilized on support 18.

As set forth in more detail herein, the methods and systems of the present disclosure can have numerous additional embodiments.

### Placement of a Peripheral Nerve on a Support

In some embodiments, the methods of the present disclosure also include a step of placing a peripheral nerve on a support (e.g., support 18 shown in FIG. 1B). In some embodiments, the placement occurs prior to electrical stimulation of the peripheral nerve. In some embodiments, the placement includes immobilization of the peripheral nerve on the support. In some embodiments, the placement of a peripheral nerve on a support can help enhance the alignment of its fascicles, which could in turn enable more effective fascicle localization.

### Electrical Stimulation

Electrical stimulation of peripheral nerves may occur in various manners. For instance, in some embodiments, electrical stimulation occurs through the utilization of one or more electrodes.

The methods and systems of the present disclosure may utilize various electrodes in various arrangements to stimulate peripheral nerves. For instance, in some embodiments, the electrodes include a plurality of electrodes. In some embodiments, the plurality of electrodes (e.g., electrodes 14 shown in FIGS. 1A-1B) are anchored to a base area (e.g., base area 12 shown in FIGS. 1A-1B). In some embodiments, the plurality of electrodes are aligned parallel to one another. In some embodiments, the plurality of electrodes are in the form of an array. In some embodiments, the plurality of electrodes span an entire width of a peripheral nerve.

In some embodiments, multiple multi-electrode arrays can be placed around the circumference of a nerve fiber and along the length of the nerve fiber to increase mapping spatial resolution. In some embodiments, multi-electrode arrays can be used to stimulate nerve fibers, record from nerve fibers, and/or measure impedances to further increase mapping spatial resolution. In some embodiments, such multi-contact electrode arrays can be fabricated using standard procedures, such as lithographic or other advanced techniques to increase the number of electrodes, which can further increase mapping spatial resolution of nerve fibers.

The methods of the present disclosure can utilize various types of electrodes for electrical stimulation. For instance, in some embodiments, the electrodes include non-intrafascicular electrodes. In some embodiments, the electrodes also serve as a sensor to sense activity.

Various methods may be utilized to electrically stimulate peripheral nerves. For instance, in some embodiments, electrical stimulation occurs by placing a plurality of electrodes (e.g., electrodes 14 shown in FIGS. 1A-1B) at different regions of the peripheral nerve, individually stimulating each of the plurality of electrodes, and detecting activity from each of the plurality of electrodes. In some embodiments, the electrodes may also detect the activity. Additionally, various stimulation waveforms and field steering techniques may be utilized to electrically stimulate peripheral nerves.

In some embodiments, a neurostimulator may be utilized for electrical stimulation of a peripheral nerve. In some embodiments, the neurostimulator may be a portable or handheld device. In some embodiments, the neurostimulator may incorporate appropriate hardware and software for safe and effective electrical charge delivery.

In some embodiments, the neurostimulator includes a computer-controlled neurostimulator. In some embodiments, the computer controlled neurostimulator may include advanced functions, such as configurations to stimulate electrodes in monopolar, bipolar, or tripolar configurations. In some embodiments, multipolar configurations may be utilized to electrically stimulate the fascicles by steering stimulation currents through them.

The methods of the present disclosure may electrically stimulate various regions of peripheral nerves. For instance, in some embodiments, the methods of the present disclosure may electrically stimulate an end organ of a peripheral nerve.

### Detected Activity

The methods of the present disclosure may be utilized to detect various types of activities from electrically stimulated peripheral nerves. For instance, in some embodiments, the detected activity is represented by a change in muscle contraction, a change in heart rate, a change in brain activity, a change in joint motion, a change in sensation, or combinations thereof.

In some embodiments, the detected activity is represented by a change in muscle contraction. In some embodiments, the change in muscle contraction is represented by a muscle twitch. In some embodiments, the change in muscle contraction is represented by a change in tibial muscle contraction, a change in peroneal muscle contraction, or combinations thereof.

In some embodiments, the detected activity is represented by a change in joint motion. In some embodiments, the change in joint motion is represented by ankle movement, ankle flexion, ankle extension, or combinations thereof.

Activities from electrically stimulated peripheral nerves may be detected in various manners. For instance, in some embodiments, the activity is detected by a method that includes, without limitation, visual detection, verbal confirmation of sensation felt, imaging, impedance spectroscopy, optical computed tomography, electromyography (EMG), joint angle kinematics, cortical recording, electroencephalography (EEG), or combinations thereof.

Activities from electrically stimulated peripheral nerves may be detected from various sources. For instance, in some embodiments, the activity may be detected from an end organ of a peripheral nerve. In some embodiments, the activity may be detected from an innervated end organ of a peripheral nerve. In some embodiments, the detected activity is detected through the utilization of one or more sensors. In some embodiments, stimulating electrodes may be used as the sensors to detect activity.

### Correlation of Detected Activity to Fascicles

Various methods may be utilized to correlate detected activity from stimulated regions of a peripheral nerve to the presence of a fascicle or a portion of a fascicle (e.g., a group of specific nerve fibers) at the stimulated regions. For instance, in some embodiments, the correlation includes correlating the detected activity to a fascicle's function (e.g., the function of a group of specific nerve fibers of a fascicle). Examples of such functions include, without limitation, muscle contraction (e.g., visible or measured), action of internal muscle contraction that leads to systemic change (e.g. urine output), elicitation of sensory responses (e.g., perceived by the individual or measured), or combinations thereof. In some embodiments, the correlation includes correlating the detected activity to a fascicle's type. For instance, in some embodiments, the detected activity is correlated to a sensory fascicle, a motor fascicle, or combinations thereof.

### Generation of a Map of Fascicles

In some embodiments, the methods of the present disclosure also include a step of generating a map of fascicles in a peripheral nerve based on the correlation. The methods of the present disclosure can be utilized to generate various types of maps. For instance, in some embodiments, the map includes a map of fascicles based on fascicle function (e.g., motor, sensory, and/or autonomic responses). In some embodiments, the map includes a map of fascicles based on fascicle type (e.g., motor, sensory, and/or mixed fascicles).

### Electrode Implantation

In some embodiments, the methods of the present disclosure may be implemented prior to an electrode implantation procedure. In some embodiments, the methods of the present disclosure also include a step of recommending electrode implantation into one or more of the located fascicles. In some embodiments, the methods of the present disclosure also include a step of implanting one or more electrodes into one or more of the located fascicles.

The methods of the present disclosure may implant various electrodes into peripheral nerves. For instance, in some embodiments, the electrodes include, without limitation, neural electrodes, intraneural electrodes, intrafascicular electrodes, or combinations thereof. In some embodiments, the electrodes include intrafascicular electrodes. In some embodiments, the intrafascicular electrodes include, without limitation, a longitudinal intrafascicular electrode (LIFE), a thin film longitudinal intrafascicular electrode (tf-LIFE), a poly longitudinal intrafascicular electrode (polyLIFE), a transverse intrafascicular multichannel electrode (TIME), a multielectrode intrafascicular array (MEA), a Utah electrode array (UEA), a Utah slanted electrode array (USEA), a distributed intrafascicular multielectrode (DIME), or combinations thereof.

The methods of the present disclosure may be utilized to implant electrodes into various regions of located fascicles. For instance, in some embodiments, the electrodes may be implanted into a nerve fiber within a fascicle.

In some embodiments, the methods of the present disclosure may be utilized to implant electrodes into located fascicles with a high level of specificity. For instance, in some embodiments, the implantation has a specificity of at least 80%. In some embodiments, the implantation has a specificity of at least 85%. In some embodiments, the implantation has a specificity of at least 90%. In some embodiments, the implantation has a specificity of at least 95%. In some embodiments, the implantation has a specificity of 100%.

### Peripheral Nerves

The methods and systems of the present disclosure may be utilized to locate a fascicle or a portion of a fascicle in various types of peripheral nerves. For instance, in some embodiments, the peripheral nerve includes, without limitation, a sensory nerve, a motor nerve, an autonomic nerve, a brachial plexus, a peroneal nerve, a femoral nerve, a lateral femoral cutaneous nerve, a sciatic nerve, a spinal accessory nerve, a tibial nerve, or combinations thereof. In some embodiments, the peripheral nerve includes a sciatic nerve.

### Modes of Operation

The methods and systems of the present disclosure may be operated in various modes. For instance, in some embodiments, fascicle localization occurs in vitro, such as in an excised nerve or an innervated tissue preparation. In some embodiments, fascicle localization occurs in vivo in a subject. In some embodiments, the subject is a human being. In some embodiments, the subject is a non-human mammal. In some embodiments, the non-human mammal includes, without limitation, a horse, a rabbit, a mouse, a rat, a pig, a sheep, a cow, a dog, or a cat. In some embodiments, the non-human mammal is a domestic animal, such as a dog, a cat, or a non-human primate.

### Applications and Advantages

The methods and systems of the present disclosure can have various advantages. For instance, by pre-mapping a fascicle's function before implanting an electrode, the methods and systems of the present disclosure provide the ability to implant an electrode in a fascicle or a portion of a fascicle that elicits a desired function.

Furthermore, in some embodiments, the methods and systems of the present disclosure can help reduce the need for implanting multiple electrodes, thereby maximizing the activation of on-target desired functions while avoiding unwanted or off-target activation. In some embodiments, the advantages of the methods and systems of the present disclosure include, without limitation: (1) an increase in specificity by pre-mapping a fascicle's function; (2) a reduction in the number of electrodes implanted that yield on-target activation and high specificity; and (3) a reduction in the occurrence of unwanted or off-target functions.

As such, the methods and systems of the present disclosure can have numerous applications. For instance, in some embodiments, the methods and systems of the present disclosure may be utilized in numerous applications to increase intrafascicular electrode implantation specificity, decrease the need for excessive intrafascicular electrode implantation, increase on-target activation, reduce off-target stimulation, or combinations thereof.

In some embodiments, the methods of the present disclosure also include a step of repairing a peripheral nerve. For instance, in some embodiments, the mapping could be utilized to repair a damaged peripheral nerve. In some embodiments, the mapping could be used to align the fascicles in repairing completely or partially lacerated nerves. In some embodiments, a step of repairing a peripheral nerve occurs independently of, or in lieu of, a step of implanting an electrode. In some embodiments, a step of repairing a peripheral nerve occurs without an electrode implantation step.

### ADDITIONAL EMBODIMENTS

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. Validation of a Fascicular Mapper for Enhancing Intraneural Electrodes Implantation Specificity

In this Example, Applicant describes the validation of a fascicular mapper for enhancing intraneural electrodes implantation specificity. In particular, Applicant has developed a system and method for implanting target-specific multiple intrafascicular electrodes in peripheral nerves to facilitate an implantation process that aims to reduce the difficulty of the implanting procedure and the time taken to implement the procedure. This Example consists of a fascicular mapper (MAP), and a method to utilize MAP during the intrafascicular electrode implantation procedure.

The MAP can pre-identify the primary function of each fascicle by electrically stimulating it and observing direct or indirect end-organ functional outcome measures. The system and method were developed to improve the effectiveness of the use of existing intrafascicular electrodes by enhancing selectivity and specificity.

Pre-mapping the fascicle's function before implanting the electrode in the fascicle provides a way to implant an electrode in a fascicle or a portion of a fascicle that elicits the desired function. This process reduces the need for implanting a large number of electrodes to yield activation of the on-target desired functions and also avoids activating unwanted or off-target activation. As such, the advantages of the MAP include, without limitation, (1) an increase in specificity by pre-mapping the fascicle's function; (2) a reduction in the number of electrodes implanted that yield on-target activation and high specificity; and (3) a reduction in the occurrence of unwanted or off-target functions.

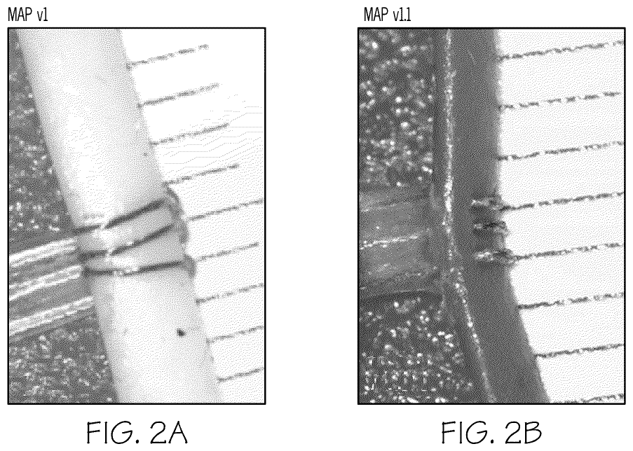

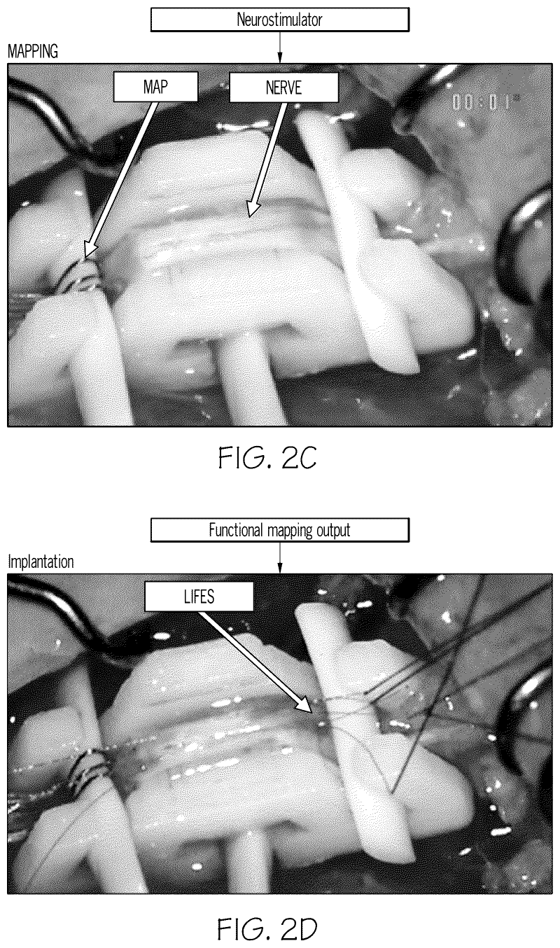

A primary function of the fascicular electrode mapper is to map the functions of the nerve fascicle or portions of the nerve fascicle by electrically stimulating it and observing end-organ functional outcome measures. The surgeon uses the pre-mapped functional outcomes as a guide to implant electrodes at the locations that elicit desired functions. The entire process for implanting target-specific LIFEs guided by the functional mapping with MAP to enhance the specificity is shown in FIGS. 2A-2D.

The ability to map fascicles in the nerve was tested in rat sciatic nerve preparation with LIFEs implanted using a custom-made platform. Hence, for proof of concept, the MAP was designed and fabricated to accommodate the LIFE implantation process with a custom-made platform. The custom-made supporting platform secures the nerve by two silicone vessel loops. During the mapping process, one of the vessel loops is replaced with the MAP, as shown in FIG. 2C.

The mapper consists of an array of non-intrafascicular electrodes (FIGS. 2A-2B) and a neurostimulator (FIG. 2C). The number, size, and material of electrodes in the mapper can be customized to the size of the nerve and the needs of the implantable neural interface.

For the proof-of-concept studies, Applicant fabricated a simple MAP prototype (FIG. 2A) with three equally spaced (˜0.5 mm apart) microwires. The number of electrodes was limited to three to span an approximately 1.5 mm diameter rat sciatic nerve. In this version of MAP (FIG. 2A), the microwires were wrapped around a silicone vessel loop and the two ends of the microwires were passed through a silicone tube. The silicone tube was pushed against the vessel loop to make microwires tightly looped around the vessel loop. A liquid super glue was injected into the silicone tube to secure the microwires around the vessel loop. After the microwires were secured, under the microscope, the microwires were de-insulated with the scalpel and fine tweezers.

In another version (FIG. 2B), instead of looping the microwires around the vessel loop, the microwires were sewn into the vessel loop for better anchoring of the electrodes and to keep the interelectrode distance constant. The mapping array can also be made using lithography technology by printing microelectrodes in any number of biocompatible substrates such as silicone, parylene, and polyimide.

Additionally, a computer-controlled neurostimulator (FIG. 2C), the arbiTrary Waveform STimulation (TWIST) system, was utilized in the mapping process. The electrodes of the mapper were stimulated one-by-one by the neurostimulator (FIG. 2C), to elicit direct outcome measures of activation of the end-organ innervated or influenced by specific nerve fascicle activation.

### Example 1.1. Animal Preparation

The fascicle mapper's mapping capabilities were evaluated in anesthetized rats (n=8; Male, Sprague Dawley, 449 (428-449) gms). The primary purpose of the acute non-survival in-vivo testing was to show that the specificity of longitudinal intrafascicular electrodes (LIFEs) implanted can be enhanced using a fascicular mapper (MAP) and allow desired on-target stimulation.

Rats were anesthetized with isoflurane (1.5-2.5% in medical-grade Oxygen) gas anesthesia. As per the IACUC guidelines, the well-being of rats was maintained by applying an ophthalmic ointment to the eyes to prevent corneal desiccation, periodically administering saline subcutaneously to prevent dehydration, and placing the body on thermal pads to prevent hypothermia. Throughout the procedure, rodent body temperature was monitored by placing a temperature probe in the rectum, and physiological indicators (respiratory rate, heart rate, and SpO₂) were monitored using a vital signs monitor (Smith Medical, Model: Surgivet® Advisor® Vital Sign Monitor, OH, USA). The level of anesthesia was periodically assessed with toe pinch, observation of eye blink, and the respiration rate.

The sciatic nerve was accessed by making a straight incision from the lateral aspect of the thigh to the ankle joint, followed by separating the biceps femoris and gluteus superficialis through minimal dissection techniques. The separated muscles were held using elastic stays and self-retaining retractors (Cooper Surgical, Model: Lone Star, CT, USA) to reach the sciatic nerve. The nerve was isolated by separating it from the surrounding connective tissue. The isolated nerve was placed on the custom-made platform.

### Example 1.2. Fascicle Mapping Process

The fascicle mapping process is shown in FIGS. 2A-2D. After placing the mapping electrode array in the reshaping slot (FIG. 2C), all the mapping electrode wires were connected to the neurostimulator. The sciatic nerve is ˜1.5 mm in diameter; hence, the three-electrode mapping array spans the entire sciatic nerve. The sciatic nerve in the rodent includes sural, peroneal, and tibial fascicles. The sural fascicle is the smallest, with a ˜200 μm diameter, and is predominantly sensory. Peroneal and tibial fascicles are ˜400 μm and 800 μm in diameter, respectively, and are predominantly motor fascicles. Stimulating a mapping electrode that is near the peroneal fascicle will elicit an ankle flexion response by contracting the tibialis anterior (TA) muscle, and near the tibial fascicle will elicit an ankle extension by contracting the extensor muscle group (Gastrocnemius (GM) and synergistic muscles). The ankle joint angle movements were observed by stimulating mapping electrodes one by one to map the motor function of the fascicles in the sciatic nerve. Before mapping, the mapping electrodes were labeled M1, M2, and M3, and after mapping, they were relabeled according to the observed motor function.

To obtain initial stimulation parameters, the PW was set at 100 μs, and PA was increased until the muscle twitch was observed at PA_{tw}. Often, muscle twitch response alone may not confirm that the primary motor function is either flexion or extension. To confirm the primary motor function, the fascicle interfacing the mapping electrode was stimulated with 1.5×PA_{tw} at 50 Hz. This stimulation parameter generates fused muscle contractions to elicit visually observable ankle joint angle movement, confirming either primarily ankle flexion or extension. This step was repeated for the remaining two mapping electrodes. The mapping electrodes that elicit ankle flexion were relabeled as MTAₓ, and the mapping electrodes that elicit ankle extension were relabeled as MGMₓ. Where x=[1, 2 or 3]. For example, if stimulation of one electrode elicits ankle flexion and two electrodes elicit ankle extension, then the electrodes will be labeled MTA₁, MGM₁, and MGM₂.

### Example 1.3. Specificity Assessment

The primary reason for mapping fascicles in this Example is to enhance the specificity of implanted LIFEs. To assess the specificity, the LIFEs were implanted in seven rats after mapping the fascicles using the mapper. The expected outcome of the LIFEs implanted in the vicinity of MTAₓ mapping electrodes will be the activation of TA. Similarly, GM activation will be expected for LIFEs implanted in the vicinity of MGMₓ mapping electrodes. The LIFE implantations were targeted depending on the need of the experimental protocols. One protocol required targeting only the tibial fascicle to activate GM. Another required targeting of both the tibial and peroneal fascicles to activate both GM and TA. During the LIFE implantation, the LIFEs expected to elicit TA and GM responses were labeled as TAₓ and GMₓ, respectively. Where x=[1, 2, . . . ] indicates LIFE numbers. After implantation, the LIFEs were stimulated to observe the elicited responses. The observed muscle response for each implanted LIFEs was recorded to assess the specificity.

### Example 1.4. Fascicle Mapping Capability Assessment

For fascicle mapping with a mapping array, the qualitative outcome measure (visual confirmation) of the observed muscle response is sufficient to determine the targeted location for implanting LIFEs. However, to validate the mapping capability, in two rats, isometric force data and in another two rats, the ankle movement video data were collected and analyzed.

### Example 1.5. Ankle Joint Force Assessment

To collect isometric force data, rats were placed laterally supporting on the left side of the rat. The rat's right paw and ankle were secured with a custom shoe designed and fabricated with a 3D printer. The 3D-printed shoe was attached to a six-degree-of-freedom force transducer (JR3, Inc., Woodland, CA, USA) for collecting isometric force data. The analog data from the force transducer were digitized using a Scout neurorecording system (Ripple Neuro, Salt Lake City, UT, USA).

In each trial, a pair of mapping electrodes (M1M2, M1M3, and M2M3) were stimulated with PWₜₕ and PAₜₕ at 10, 30, and 50 Hz. The stimulation sequence for each electrode was burst of stimulation ON for ˜500 ms followed by stimulation OFF for ˜500 ms. Force data generated from twenty stimulation sequences for each combination of electrode pairs were collected and analyzed. The ON and OFF stimulation time was inconsistent because of software-controlled timing process and over USB communication instead of hardware control.

### Example 1.6. Ankle Joint Angle Assessment

During the fascicle mapping process, one electrode (M1, M2, or M3) that elicited ankle flexion and another electrode that elicited ankle extension were selected. For each selected electrode, the twitch threshold (PA_{tw}) at 100 Hz stimulation frequency was determined by gradually increasing the PA until muscle twitch was elicited. The video data were collected for each electrode separately using a handheld portable video camera while stimulating with 1.5×PA_{tw} at 50 Hz for ˜500 ms. The 50 Hz stimulation frequency was chosen to generate fused muscle contractions for offline video analysis.

### Example 1.7. Specificity Assessment

The expected and observed outcomes for all implanted LIFEs using the mapping process were tabulated for cach animal experiment and in Table 1.

TABLE 1
Specificity assessment. In five out of seven rats, 100% specificity was achieved.

| | Tibial fascile | | Peroneal fascile | | | |
|---|---|---|---|---|---|---|
| | (# of LIFEs) | | (# of LIFEs) | | Specificity | |
| Rat # | Expected | Observed | Expected | Observed | Tibial | Peroneal |
| UA011 | 3 | 3 | 2 | 2 | 100% | 100% |
| UA012 | 5 | 5 | | | 100% | |
| UA018 | 1 | 1 | 1 | 1 | 100% | 100% |
| UA020 | 3 | 3 | 1 | 1 | 100% | 100% |
| UA022 | 5 | 4 | 0 | 1 | 80% | |
| UA023 | 5 | 4 | 0 | 1 | 80% | |
| UA024 | 3 | 3 | 2 | 2 | 100% | 100% |

The specificity of the LIFEs implanted in the tibial and peroneal fascicle for each rat was calculated as the ratio of the number of LIFEs whose expected and observed outcomes matched the total number of electrodes implanted. If the observed outcomes for all the LIFEs implanted in the fascicle were as expected, then the achieved specificity will be 100%.

A confusion matrix was constructed to calculate the overall accuracy, tibial specificity, and peroneal specificity. The accuracy is the ratio of the number of the LIFEs matched for both tibial and peroneal fascicles to the total number of the LIFEs implanted. The sensitivity for each fascicle is the ratio of the LIFEs matched for that fascicle to the expected number of LIFEs implanted.

### Example 1.8. Fascicle Mapping Capability Assessment

A suite of custom Matlab® routines, including modifications to application interface (API) routines provided by the neurorecording system manufacturer, was developed to extract, transform, process, and analyze the force data. Other data analyses platforms could also be utilized. To remove high-frequency noise, the raw data were low-pass filtered with a 4^{th}-order Butterworth filter at 50 Hz. The filter was applied twice (once forward and then backward) to reduce the phase difference.

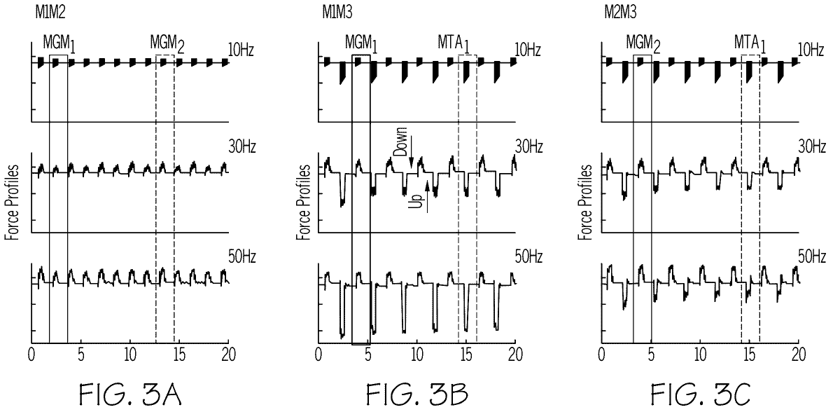

The captured video data was imported into validated Kinovea (kinovea.org) kinematic analysis software. The ankle angle was defined by three markers placed on the rat hindlimb. The markers in the video were digitized to obtain ankle angle data and exported to Matlab® to plot (FIGS. 3A-3C).

The fascicle mapper prototypes fabricated consisted of an electrode array with three equally spaced (˜0.5 mm apart) electrodes (M1, M2, and M3) that spanned the entire width of the rat's sciatic nerves, which were ˜1.5 mm in diameter. Because the tibial fascicle is the largest (˜800 μm), at least two electrodes interfaced with the tibial fascicle, and one electrode interfaced with the peroneal fascicle.

### Example 1.8.1. Specificity Assessment

Specificity is achieved when the observed function of the implanted LIFEs matched the expected function. Table 1 shows the results from the mapping process in seven rats. In five out of seven rats, 100% specificity was achieved for both tibial and peroneal fascicles. In three rats, the targeted fascicle was the tibial fascicle, and with the mapping process, the specificity achieved was 100% in one and 80% in two rats. The tibial and peroneal fascicles were targeted in four rats, and a 100% specificity was achieved through the mapping process.

The constructed confusion matrix and the computed performance metrics from the mapping data are shown in Tables 2 and 3, respectively.

TABLE 2
Confusion matrix from mapping data.

| | Tibial (Expected) | Peroneal (Expected) |
|---|---|---|
| Tibial (Observed) | 23 | 0 |
| Peroneal (Observed) | 2 | 6 |

TABLE 3
Performance metrics from mapping data.

| Performance Metrics | % |
|---|---|
| Overall accuracy | 94% |
| Tibial specificity | 92% |
| Peroneal specificity | 100% |

The overall mapping accuracy from all the LIFEs implanted is 94%. The specificities achieved for the LIFEs implanted in tibial and peroneal fascicles are 92% and 100%. More LIFEs were implanted in the tibial fascicle than the peroneal fascicle since the tibial fascicle is larger in diameter than the peroneal fascicle.

### Example 1.9. Fascicle Mapping Capability Assessment-Ankle Joint Force Assessment

The isometric force from alternate stimulation of mapping electrode pairs is shown in FIGS. 3A-3C. The ankle extension response (FIGS. 3A and 3C, Down (Push force)) can be observed from isometric force data generated by stimulating the fascicle or portion of fascicle interfacing mapping electrodes M1 and M2 and are marked as MGM₁ and MGM₂. Similarly, the M3 mapping electrode shows ankle flexion (FIGS. 3B and 3C: Up (Pull force)) and is marked as MTA₁. Therefore, the results indicate that the fascicle or portion of fascicle interfacing M1 and M2 mapping electrodes is tibial, and M3 is a peroneal fascicle. In addition, comparing the results between electrode pairs M1M2 (FIG. 3A), M1M3 (FIG. 3B), and M2M3 (FIG. 3C), it can be observed that M1 is primarily aligned with the tibial fascicle and M3 with the peroneal fascicle. Thus, to enhance the specificity for ankle extension, the LIFEs should be implanted into the nerve tissue aligned with mapping electrode M1. Similarly, to enhance the specificity for ankle flexion, the LIFEs should be implanted along the direction of mapping electrode M3.

The results for increasing stimulation frequency follow a typical trend: increased force with increased stimulation frequency. As expected, the contractions were not fused (FIGS. 3A-3C, Panel 1) for 10 Hz stimulation frequency and were completely fused for 50 Hz stimulation frequency (FIG. 3A, Panel 3).

### Example 1.10. Fascicle Mapping Capability Assessment-Ankle Joint Ankle Assessment

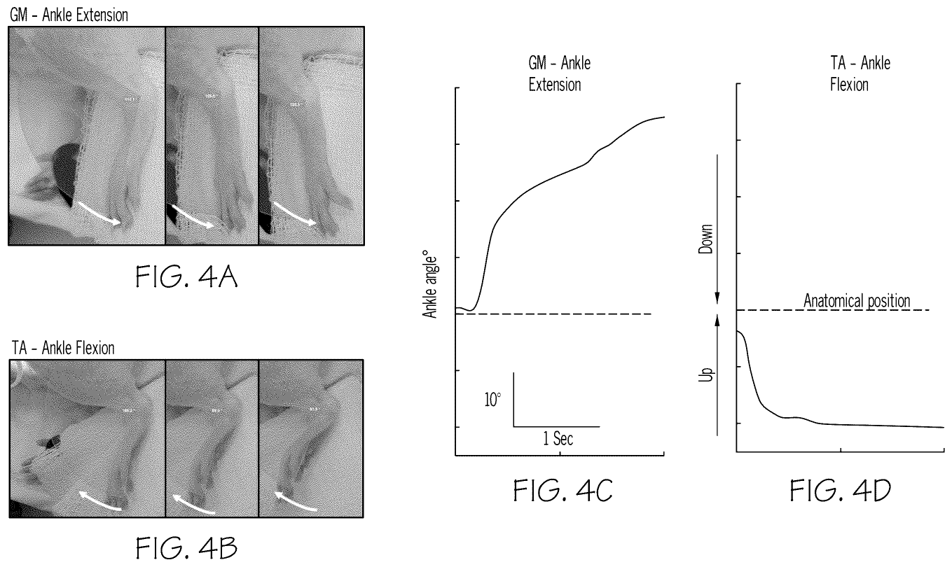

The video data collected during the fascicle mapping process show that stimulation of two mapping electrodes elicits ankle extension (FIG. 4A) and ankle flexion (FIG. 4B) movements. The ankle flexion is a decreasing angle from the anatomical position denoted by a broken line shown in FIG. 4C. An increasing angle from the anatomical position is defined as ankle extension (FIG. 4D). These results show that the mapping electrode elicited ankle flexion when interfaced with the peroneal fascicle, and the mapping electrode elicited ankle flexion when interfaced with the tibial fascicle.

In sum, Applicant has developed a system and method for implanting target-specific multiple intrafascicular electrodes to enhance selectivity and specificity of intraneural electrodes. This system and method reduce the need for implanting a large number of electrodes in order to achieve the desired functions and also avoids activating unwanted or off-target functions.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein

1. A method of locating a fascicle or a portion of a fascicle of a peripheral nerve, said method comprising:
electrically stimulating different regions of the peripheral nerve;
detecting activity from the stimulated regions; and
correlating the detected activity from the stimulated regions to the presence of a fascicle or a portion of a fascicle at the stimulated regions.

2. The method of claim 1, further comprising a step of placing the peripheral nerve on a support prior to the electrical stimulation.

3. The method of claim 1, wherein the electrical stimulation occurs by placing a plurality of electrodes at different regions of the peripheral nerve, individually stimulating each of the plurality of electrodes, and detecting activity from each of the plurality of electrodes.

4. The method of claim 3, wherein the plurality of electrodes are aligned parallel to one another.

5. The method of claim 3, wherein the plurality of electrodes span an entire width of the peripheral nerve.

6. The method of claim 1, wherein the detected activity is represented by a change in muscle contraction, a change in heart rate, a change in brain activity, a change in joint motion, a change in sensation, or combinations thereof.

7. The method of claim 1, wherein the detected activity is detected by a method selected from the group consisting of visual detection, verbal confirmation of sensation felt, imaging, impedance spectroscopy, optical computed tomography, electromyography (EMG), joint angle kinematics, cortical recording, electroencephalography (EEG), or combinations thereof.

8. The method of claim 1, wherein the correlating comprises correlating the detected activity to a fascicle's function.

9. The method of claim 1, wherein the correlating comprises correlating the detected activity to a fascicle's type.

10. The method of claim 9, wherein the detected activity is correlated to a sensory fascicle, a motor fascicle, or combinations thereof.

11. The method of claim 1, further comprising a step of generating a map of fascicles in the peripheral nerve based on the correlating.

12. The method of claim 11, wherein the map comprises a map of fascicles based on fascicle function.

13. The method of claim 11, wherein the map comprises a map of fascicles based on fascicle type.

14. The method of claim 1, further comprising a step of implanting one or more electrodes into one or more of the located fascicles.

15. The method of claim 1, wherein the peripheral nerve is selected from the group consisting of a sensory nerve, a motor nerve, an autonomic nerve, a brachial plexus, a peroneal nerve, a femoral nerve, a lateral femoral cutaneous nerve, a sciatic nerve, a spinal accessory nerve, a tibial nerve, or combinations thereof.

16. The method of claim 1, wherein the peripheral nerve comprises a sciatic nerve.

17. The method of claim 1, wherein the method occurs in vitro.

18. The method of claim 1, wherein the method occurs in vivo in a subject.

19. The method of claim 18, wherein the subject is a human being.

20. The method of claim 1, further comprising a step of repairing the peripheral nerve.

21. A system for mapping a peripheral nerve, wherein the system comprises:
a plurality of electrodes, wherein each of the plurality of electrodes is operational to be actuated independently of other electrodes; and
a base area, wherein the base area anchors the plurality of electrodes.

22. The system of claim 21, further comprising a wiring system, wherein the wiring system connects the plurality of electrodes to a power source.

23. The system of claim 22, wherein the power source comprises a neural stimulator.

24. The system of claim 21, wherein the plurality of electrodes are aligned parallel to one another.

25. The system of claim 21, wherein the electrodes are associated with the base area in the form of an array.

26. The system of claim 21, wherein the electrodes are fully looped around the base area.
